(19) United States
(12) Patent Application Publication
(10) Pub. No.: US 2005/0079588 A1
(43) Pub. Date: Apr. 14, 2005

(54) Method for the fermentative production of L-amino acids, using coryneform bacteria

(75) Inventors: Georg Sindelar, Kaarst (DE); Volker F. Wendisch, Juelich (DE); Hermann Sahm, Juelich (DE)

Correspondence Address: OBLON, SPIVAK, MCCLELLAND, MAIER & NEUSTADT, P.C., 1940 DUKE STREET, ALEXANDRIA, VA, 22314, US

(73) Assignees: DEGUSSA AG, Duesseldorf (DE); FORSCHUNGSZENTRUM JUELICH GMBH, Juelich (DE)

(21) Appl. No.: 10/941,920

(22) Filed: Sep. 16, 2004

(30) Foreign Application Priority Data
Sep. 26, 2003 (DE) 103 44 739.3

(51) Int. Cl. C12P 13/08; C12N 15/74; C12N 1/21

(52) U.S. Cl. 435/115; 435/252.3; 435/471

(57) ABSTRACT

L-amino acid is produced by fermenting a medium using coryneform bacteria in which one or more of the genes linked with a nitrogen metabolism and selected from the group consisting of amt, ocd, soxA and sumT is/are amplified.

### BACKGROUND OF THE INVENTION

1. Field of the Invention

The present the invention relates to a method for the production of a L-amino acid by fermentation using coryneform bacteria, in which one or more of the genes, selected from the group consisting of amt, ocd, soxA and sumT is/are amplified.

2. Description of the Related Art

Chemical compounds, which term is particularly meant to refer to L-amino acids, vitamins, nucleosides and nucleotides and D-amino acids, are used in human medicine, in the pharmaceutical industry, in cosmetics, in the foods industry, and in animal nutrition.

Numerous of these compounds are produced by means of fermentation of strains of coryneform bacteria, particularly Corynebacterium glutamicum. Because of their great importance, work is constantly being done to improve the production methods. Method improvements can relate to measures of fermentation technology, such as stirring and supplying oxygen; to the composition of the nutrient media, such as the sugar concentration during fermentation; to the processing to produce the product form, by means of ion exchange chromatography, for example, or to the intrinsic performance properties of the microorganism itself.

In order to improve the performance properties of these microorganisms, methods of mutagenesis, selection, and mutant selection are used. In this manner, strains are obtained that are resistant against anti-metabolites such as the lysine analog S-(2-aminoethyl)-cysteine, for example, or are auxotrophic for metabolites that are significant for regulation, and produce L-amino acids.

For some years, methods of recombinant DNA technology have also been used to improve strains of Corynebacterium glutamicum that produce L-amino acids, in that individual amino acid synthesis genes are amplified and the effect on the L-amino acid production is examined.

### SUMMARY OF THE INVENTION

It is an object of the present invention to provide a method for the fermentative production of L-amino acids, particularly L-lysine, using coryneform bacteria.

It is a further object to use in the above method coryneform bacteria which already produce L-amino acids and in which at least one or more of the nucleotide sequence(s) that code(s) for the genes amt, ocd, soxA and/or sumT is/are amplified, particularly over-expressed or expressed on a high level.

Even further, it is an object to use coryneform bacteria which already produce L-amino acids, particulalrly L-lysine, even before amplification of one or more of the genes amt, ocd, soxA and/or sumT.

Another object of the present invention is to provide the microorganisms used for the fermentation.

This and other objects have been achieved by the present invention the first embodiment of which includes a method for producing an L-amino acid, comprising:

fermenting a medium using coryneform bacteria in which one or more of the genes linked with a nitrogen metabolism and selected from the group consisting of amt, ocd, soxA and sumT is/are amplified.

In another embodiment, the present invention relates to a method for producing an L-amino acid, comprising:

a) fermenting a medium using recombinant coryneform bacteria that produce said L-amino acid,
wherein in said bacteria at least one or more of the genes selected from the group consisting of amt, ocd, soxA and sumT is/are amplified;

b) accumulating said L-amino acid in said medium or in the cells of said bacteria, and

c) isolating said L-amino acid.

In yet another embodiment, the present invention relates to coryneform bacteria in which at least one or more of the genes selected from the group consisting of amt, ocd, soxA and sumT is/are present in amplified form.

### BRIEF DESCRIPTION OF DRAWINGS

FIG. 1 shows a map of the plasmid pVWEx1_mt_ocd_soxA.

FIG. 2 shows a map of the plasmid pVWEx1_sumT.

### DETAILED DESCRIPTION OF THE INVENTION

When L-amino acids or amino acids are mentioned hereinafter, this refers to one or more of the proteinogenic amino acids, including their salts, selected the group consisting of L-asparaginic acid, L-asparagine, L-threonine, L-serine, L-glutamic acid, L-glutamine, L-glycine, L-alanine, L-cysteine, L-valine, L-methionine, L-isoleucine, L-leucine, L-tyrosine, L-phenylalanine, L-histidine, L-tryptophan, L-arginine, and L-proline. L-lysine is particularly preferred.

Proteinogenic amino acids are understood to be the amino acids that occur in natural proteins, in other words in proteins of microorganisms, plants, animals, and humans.

When L-lysine or lysine are mentioned hereinafter, this refers not only to the bases, but also to the salts, such as lysine monohydrochloride or lysine sulfate, for example.

The present invention relates to a method for the fermentative production of L-amino acids, using coryneform bacteria, which particularly already produce L-amino acids and in which at least one or more of the nucleotide sequence(s) that code(s) for the genes amt, ocd, soxA and/or sumT is/are amplified, particularly over-expressed or expressed on a high level.

Furthermore, the present invention relates to a method for the fermentative production of L-amino acids, comprising:

a) fermentation of a medium by the coryneform bacteria, preferably recombinant bacteria, that produce L-amino acid, in which at least one or more of the genes, selected from the group consisting of amt, ocd, soxA and/or sumT, is/are amplified, particularly over-expressed or expressed on a high level,

b) accumulation of the L-amino acids in the medium or in the cells of the bacterium, and

c) isolation of the desired L-amino acids, whereby if applicable, residues of the fermentation liquid and/or the biomass remain in the end product, in portions (>0 to 100%, preferably <100%) or in their total amount.

The coryneform bacteria used preferably produce L-amino acids, more preferably L-lysine, even before amplification of one or more of the genes amt, ocd, soxA and/or sumT. It was found that these coryneform bacteria produce L-amino acids, particularly L-lysine, in an improved manner after amplification of one or more of the genes amt, ocd, soxA and/or sumT.

The gene products of the three genes amt, ocd and soxA, arranged in an operon, are linked with the nitrogen metabolism.

The gene amt codes for the ammonium transporter Amt in Corynebacterium glutamicum (Siewe et al., Journal of Biological Chemistry 271 (10): 5398-5402 (1996)), which is expressed as a function of the internal glutamine, glutamine analog, and NH₄⁺ concentration, and transports (methyl) ammonium into the cell, driven by protons (Meier-Wagner et al., Microbiology 147 (Pt 1): 135-143 (2001)).

The gene soxA codes for a sarcosine oxidase (Siewe et al., Journal of Biological Chemistry 271 (10): 5398-5402 (1996)). Sarcosine oxidases belong to a group of oxidases containing flavine, which catalyze oxidative reactions with tertiary and secondary amino acids and also release ammonium by means of deamination in Bacillus subtilis and Corynebacterium sp. P-1 (Job et al., Journal of Biological Chemistry 277 (9): 6985-6993 (2002); Chlumsky et al., Biochemistry 32 (41): 11132-11142 (1993)).

The gene ocd codes for an ornithine cyclodeaminase (Jakoby et al, FEMS Microbiology Letters 173 (2): 303-310 (1999)). Ornithine cyclodeaminases catalyze the decomposition of citrulline and arginine to ornithine in pseudomonas, and thereby release ammonium in the form of urea or carbamoyl phosphate (Stalon et al., Journal of General Microbiology 133 (PT9): 2487-2495 (1987)).

The gene sumT codes for a methyl transferase from a group of uroporphyrine-III-C-methyl transferases (EC: 2.1.1.107), which catalyzes the transfer of two methyl groups from S-adenosyl methionine to uroporphyrinogen III. The product precorrin-2 is an intermediate in the biosynthesis of corrinoids such as cobalamine (Vitamin B 12), sirohem, hemd, or coenzyme F430 (Raux et al., Cell Molecular Live Science 57 (13-14): 1880-1893 (2000)).

The nucleotide sequences of the said genes of Corynebacterium glutamicum belong to the state of the art and can be found in various publications, patent applications, as well as the database of the National Center for Biotechnology Information (NCBI) of the National Library of Medicine (Bethesda, Md., USA).

| | |
|---|---|
| amt gene: | |
| Designation: | ammonium transporter Amt |
| References: | Siewe et al., Journal of Biological Chemistry 271 (10): |
| | 5398-5403 (1996); sequences No. 3468 and |
| | No. 7064 from EP1108790 |
| Accession No.: | X93513, AX123552, AX127148, and AJ007732 |
| ocd gene: | |
| Designation: | putative ornithine cyclodeaminase Ocd |
| References: | sequences No. 3467 and No. 7064 from EP1108790 |
| Accession No.: | AX123551, AX127148, and AJ007732 |
| soxA gene: | |
| Designation: | sarcosine oxidase SoxA |
| References: | sequences No. 1748 and No. 7064 from EP1108790 |
| Accession No.: | AX121832, AX127148, and AJ007732 |
| sumT: | |
| Designation: | methyl transferase SumT |
| References: | sequences No. 994 and No. 7062 from EP1108790 |
| Accession No.: | AX121978 and AX127146. |

The sequences described in the above texts, coding for the genes amt, ocd, soxA and/or sumT, can be used according to the present invention. Furthermore, alleles of the said genes can be used, which result from the degeneracy of the genetic code or by means of function-neutral sense mutations.

In this connection, the term “amplification” or “amplify” describes the increase in intracellular activity or concentration of one or more enzymes or proteins in a microorganism, which are coded by the corresponding DNA, in that the number of copies of the gene or genes is increased, for example, a strong promoter or a gene or allele is used that codes for a corresponding enzyme or protein having a high activity or, if applicable, these measures are combined.

By means of the measures of amplification, particularly over-expression, the activity or concentration of the corresponding protein is generally increased by at least 10%, 25%, 50%, 75%, 100%, 150%, 200%, 300%, 400% or 500%, maximally up to 1000% or 2000%, with reference to the wild type of protein, i.e. with reference to the activity or concentration of the protein in the starting microorganism. Thus, the activity or concentration of the corresponding protein is generally increased by 10-2000%, with reference to the wild type of protein.

The increase in protein concentration can be detected in the gel by way of one-dimensional and two-dimensional protein gel separation and subsequent optical identification of the protein concentration, using corresponding evaluation software. A common method for the preparation of the protein gels in the case of coryneform bacteria and for the identification of the proteins is the method of procedure described by Hermann et al. (Electrophoresis, 22: 1712-23 (2001)). The protein concentration can also be analyzed by means of Western blot hybridization with an antibody specific for the protein to be detected (Sambrook et al., Molecular Cloning: A Laboratory Manual. 2nd edition, Cold Spring Harbor Laboratory Press, Cold Spring Harbor, N.Y., 1989), and subsequent optical evaluation using corresponding software for determining the concentration (Lohaus and Meyer (1998), Biospektrum [Biospectrum] 5: 32-39; Lottspeich (1999), Angewandte Chemie [Applied Chemistry] 111: 2630-2647). The activity of DNA-binding proteins can be measured by means of DNA band shift assays (also referred to as gel retardation) (Wilson et al. (2001), Journal of Bacteriology 183: 2151-2155). The effect of DNA-binding proteins on the expression of other genes can be determined by means of various methods of the reporter gene assay, which have been well described (Sambrook et al., Molecular Cloning: A Laboratory Manual. 2nd edition, Cold Spring Harbor Laboratory Press, Cold Spring Harbor, N.Y., 1989).

The microorganisms used in the present invention can produce amino acids from glucose, saccharose, lactose, fructose, maltose, molasses, starch, cellulose, or from glycerin and ethanol. The above compounds can be part of a medium or be used alone as the medium. The microorganisms can be representatives of coryneform bacteria, particularly of the genus Corynebacterium. In the case of the genus Corynebacterium, the species Corynebacterium glutamicum should be particularly mentioned, which is known in the art for its ability to produce L-amino acids.

Suitable strains of the genus Corynebacterium, particularly of the species Corynebacterium glutamicum, are particularly the known wild type strains

Corynebacterium glutamicum ATCC13032,

Corynebacterium acetoglutamicum ATCC15806,

Corynebacterium acetoacidophilum ATCC13870,

Corynebacterium melassecola ATCC 17965,

Corynebacterium thermoaminogenes FERM BP-1539,

Brevibacterium flavum ATCC14067,

Brevibacterium lactofermentum ATCC13869, and

Brevibacterium divariticum ATCC14020,

and mutants or strains that produce L-amino acids, produced from these, such as, for example, the strains that produce L-lysine

Corynebacterium glutamicum FERM-P 1709,

Brevibacterium flavum FERM-P 1708,

Brevibacterium lactofermentum FERM-P 1712,

Corynebacterium glutamicum FERM-P 6463,

Corynebacterium glutamicum FERM-P 6464, and

Corynebacterium glutamicum DSM 5715.

Strains with the designation “ATCC” can be purchased from the American Type Culture Collection (Manassas, Va., USA). Strains with the designation “FERM” can be purchased from the National Institute of Advanced Industrial Science and Technology (AIST Tsukuba Central 6, 1-1-1 Higashi, Tsukuba Ibaraki, Japan). The listed strain of Corynebacterium thermoaminogenes (FERM BP-1539) is described in U.S. Pat. No. b 5,250,434.

In order to achieve over-expression, the number of copies of the corresponding genes can be increased, or the promoter and regulation region or the ribosome binding location, which is located upstream of the structure gene, can be mutated. Expression cassettes, which are built in upstream of the structure gene, act in the same manner. It is additionally possible, by means of inducible promoters, to increase the expression in the course of the fermentative amino acid production. The expression is also improved by means of measures to lengthen the lifetime of the m-RNA. Furthermore, the enzyme activity is increased by means of preventing the decomposition of the enzyme protein. The genes or gene constructs can be present either in plasmids having different numbers of copies, or can be integrated into the chromosome and amplified. Alternatively, an over-expression of the genes in question can furthermore be achieved by means of changing the composition of the medium and the way in which culturing is conducted.

Instructions in this regard are found, by a person skilled in the art, in Martin et al. (Bio/Technology 5, 137-146 (1987)), in Guerrero et al. (Gene 138, 35-41 (1994)), Tsuchiya and Morinaga (Bio/Technology 6, 428-430 (1988)), in Eikmanns et al. (Gene 102, 93-98 (1991)), in the European patent 0 472 869, in the U.S. Pat. No. 4,601,893, in Schwarzer and Pühler (Bio/Technology 9, 84-87 (1991)), in Reinscheid et al. (Applied and Environmental Microbiology 60, 126-132 (1994)), in LaBarre et al. (Journal of Bacteriology 175, 1001-1007 (1993)), in the patent application WO 96/15246, in Malumbres et al. (Gene 134, 15-24 (1993)), in the Japanese published patent application JP-A-10-229891, in Jensen and Hammer (Biotechnology and Bioengineering 58, 191-195 (1998)), in Makrides (Microbiological Reviews 60: 512-538 (1996)), and in known texts relating to genetics and molecular biology, among others.

For amplification, one or more of the genes, selected from the group consisting of amt, ocd, soxA, and sumT, was/were over-expressed using episomal plasmids, as an example. Suitable plasmids are those that are replicated in coryneform bacteria. Numerous known plasmid vectors, such as pZ1 (Menkel et al., Applied and Environmental Microbiology (1989), 64: 549-554), pEKEx1 (Eikmanns et al., Gene 102: 93-98 (1991)), or PHS2-1 (Sonnen et al., Gene 107: 69-74 (1991)), for example, are based on the cryptic plasmids pHM1519, pBL1, or pGA1. Other plasmid vectors such as those that are based on pCG4 (U.S. Pat. No. 4,489,160), or pNG2 (Serwold-Davis et al., FEMS Microbiology Letters 66, 119-124 (1990)), or pAG1 (U.S. Pat. No. 5,158,891), for example, can be used in the same manner.

Furthermore, those plasmid vectors with which one can use the method of gene amplification by means of integration into the chromosome, as described by Reinscheid et al. (Applied and Environmental Microbiology 60, 126-132 (1994)) for the duplication or amplification of the hom-thrB operon, for example, are also suitable. In this method, the complete gene is cloned into a plasmid vector, which can replicate in a host (typically E. coli), but not in C. glutamicum. Possible vectors are, for example, pSUP301 (Simon et al., Bio/Technology 1, 784-791 (1983)), pK18mob or pK19mob (Schäfer et al., Gene 145, 69-73 (1994)), pGEM-T (Promega Corporation, Madison, Wis., USA), pCR2.1-TOPO (Shuman (1994)), Journal of Biological Chemistry 269: 32678-84; U.S. Pat. No. 5,487,993), pCR®Blunt (Invitrogen Company, Groningen, the Netherlands; Bernard et al., Journal of Molecular Biology, 234: 534-541 (1993)), pEM1 (Schrumpfet al., 1991, Journal of Bacteriology 173: 4510-4516), or pBGS8 (Spratt et al., 1986, Gene 41: 337-342). The plasmid vector that contains the gene to be amplified is subsequently transformed to the desired strain of C. glutamicum by means of conjugation or transformation. The method of conjugation is particularly described in Schäfer et al. (Applied and Environmental Microbiology 60, 756-759 (1994)), for example. Methods for transformation are described, for example, in Thierbach et al. (Applied Microbiology and Biotechnology 29, 356-362 (1988)), Dunican and Shivnan (Bio/Technology 7, 1067-1070 (1989)), and Tauch et al. (FEMS Microbiology Letters 123, 343-347 (1994)). After homologous recombination by means of a “cross-over” event, the resulting strain contains at least two copies of the gene in question.

A common method for building one or more additional copies of a gene of C. glutamicum into the chromosome of the desired coryneform bacterium is the method of gene doubling described in Schwarzer and Pühler (Bio/Technology 9, 84-87 (1991)), Peters-Wendisch et al. (Microbiology 144, 915-927 (1998)), as well as in WO 03/014330 and WO 03/04037. For this purpose, the nucleotide sequence of the desired ORF, gene, or allele, if applicable including the expression and/or regulation signals, is isolated, and two copies, preferably in a tandem arrangement, are cloned in a vector that is not replicative for C. glutamicum, such as pK18mobsacB or pK19mobsacB, for example (Jäger et al., Journal of Bacteriology 174: 5462-65 (1992)). The vector is subsequently transformed into the desired coryneform bacterium by means of transformation or conjugation. After homologous recombination by means of a first “cross-over” event that causes integration, and a suitable second “cross-over” event that causes an excision, in the target gene or in the target sequence, building in the mutation takes place. Afterwards, those bacteria in which two copies of the ORF, gene or allele are present at the natural location, instead of the originally present singular copy, are isolated. In this connection, no nucleotide sequence that is enabled for or enables episomal replication in microorganisms, no nucleotide sequence that is enabled for or enables transposition, and no nucleotide sequence that imparts resistance against antibiotics remains at the natural gene location, in each instance.

In addition, it can be advantageous for the production of L-amino acids either to amplify, particularly to over-express, one or more enzymes of the biosynthesis path, in each instance, of glycolysis, or anaplerotics, of the citric acid cycle, of the pentose phosphate cycle, of amino acid export and, if applicable, regulatory proteins, in addition to amplification of one or more of the genes selected from the group consisting of amt, ocd, soxA and/or sumT, or to weaken them, particularly to reduce the expression.

In this connection, the term “weakening” describes the reduction or shut-off of the intracellular activity of one or more enzymes (proteins) in a microorganism, which are coded by the corresponding DNA, in that a weak promoter is used, for example, or a gene or allele is used that codes with a low activity for a corresponding enzyme or protein, or that inactivates the gene or enzyme (protein) in question and, if applicable, combines these measures.

By means of the measures of weakening, the activity or concentration of the corresponding protein is generally lowered to 0 to 75%, 0 to 50%, 0 to 25%, 0 to 10%, or 0 to 5% of the activity or concentration of the wild type protein, i.e. the activity or concentration of the protein in the starting organism.

The use of endogenic genes is generally preferred. “Endogenic genes” or “endogenic nucleotide sequences” are understood to mean the genes or nucleotide sequences that are present in the population of a species.

Thus, for example, for the production of L-lysine, in addition to amplification of one or more of the genes selected from the group consisting of amt, ocd, soxA and/or sumT, one or more of the genes selected from the group consisting of the genes or alleles for lysine production can be amplified, particularly over-expressed. “Gene or allele of lysine production” is understood to mean all the, preferably endogenic, open read frames, genes, or alleles whose amplification/over-expression can result in an improvement of lysine production.

These include, among others, the following genes or alleles: accBC, accDA, cstA, cysD, cysE, cysH, cysK, cysN, cysQ, dapA, dapB, dapC, dapD, dapE, dapF, ddh, dps, eno, gap, gap2, gdh, gnd, lysC, lysCFBR, lysE, msiK, opcA, oxyR, ppc, ppcFBR, pgk, pknA, pknB, pknD, pknG, ppsA, ptsH, ptsI, ptsM, pyc, pyc Pro458Ser, sigc, sigD, sigE, sigH, sigM, ta1, thyA, tkt, tpi, zwa1, zwf, and Ala213Thr. These are listed and explained in Table 1.

TABLE 1
Genes and alleles of lysine production

| | Designation of the coded enzyme or | | Access |
|---|---|---|---|
| Name | protein | Reference | Number |
| accBC | acyl-CoA carboxylase | Jäger et al. | 035023 |
| | EC 6.3.4.14 | Archives of | |
| | | Microbiology | |
| | | (1996) 166: 76-82; | |
| | | EP1108790; | AX123524 |
| | | WO0100805 | AX066441 |
| accDA | acetyl-CoA carboxylase | EP1055725 | |
| | EC 6.4.1.2 | EP1108790 | AX121013 |
| | | WO0100805 | AX066443 |
| cstA | carbon starvation protein A | EP1108790 | AX120811 |
| | | WO0100804 | AX066109 |
| cysD | sulfat-adenylyltransferase subunit II | EP1108790 | AX123177 |
| | EC 2.7.7.4 | | |
| cysE | serine acetyltransferase | EP1108790 | AX122902 |
| | EC 2.3.1.30 | WO0100843 | AX063961 |
| cysH | 3′-phosphoadenosine 5′-phosphosulfate | EP1108790 | AX123178 |
| | reductase | WO0100842 | AX066001 |
| | EC 1.8.99.4 | | |
| cysK | cysteine synthase | EP1108790 | AX122901 |
| | EC 4.2.99.8 | WO0100843 | AX063963 |
| cysN | sulfate adenylyltransferase subunit I | EP1108790 | AX123176 |
| | EC 2.7.7.4 | | AX127152 |
| cysQ | transporter protein cysQ | EP1108790 | AX127145 |
| | | WO0100805 | AX066423 |
| dapA | dihydrodipicolinate synthase | Bonnassie et al. | X53993 |
| | EC 4.2.1.52 | Nucleic Acids | |
| | | Research 18: 6421 | |
| | | (1990); | |
| | | Pisabarro et al., | Z21502 |
| | | Journal of | |
| | | Bacteriology | |
| | | 175: 2743-2749 | |
| | | (1993); | |
| | | EP1108790; | |
| | | WO0100805; | |
| | | EP0435132; | |
| | | EP1067192; | |
| | | EP1067193; | AX123560 |
| | | | AX063773 |
| dapB | dihydrodipicolinat reductase | EP108790 | AX127149 |
| | EC 1.3.1.26 | WO0100843 | AX063753 |
| | | EP1067192 | AX137723 |
| | | EP1067193 | AX137602 |
| | | Pisabarro et al., | X67737 |
| | | Journal of | Z21502 |
| | | Bacteriology | |
| | | 175: 2743-2749 | |
| | | (1993) | |
| | | JP1998215883 | |
| | | JP1997322774 | E16749 |
| | | JP1997070291 | E14520 |
| | | JP1995075578 | E12773 |
| | | | E08900 |
| dapC | N-succinyl-diaminopimelate transaminase | EP1108790 | AX127146 |
| | EC 2.6.1.17 | WO0100843 | AX064219 |
| | | EP1136559 | |
| dapD | tetrahydrodipicolinat succinylase EC | EP1108790 | AX127146 |
| | 2.3.1.117 | WO0100843 | AX063757 |
| | | Wehrmann et al. | AJ004934 |
| | | Journal of | |
| | | Bacteriology | |
| | | 180: 3159-3165 | |
| | | (1998) | |
| dapE | N-succinyl-diaminopimelate | EP1108790 | AX127416 |
| | desuccinylase | WO0100843 | AX063749 |
| | EC 3.5.1.18 | Wehrmann et al. | X81379 |
| | | Microbiology | |
| | | 140: 3349-3356 | |
| | | (1994) | |
| dapF | diaminopimelate epimerase | EP1108790 | AX127149 |
| | EC 5.1.1.7 | WO0100843 | AX063719 |
| | | EP1085094 | AX137620 |
| Ddh | diaminopimelate dehydrogenase | EP1108790 | AX127152 |
| | EC 1.4.1.16 | WO0100843 | AX063759 |
| | | Ishino et al., | Y00151 |
| | | Nucleic Acids | |
| | | Research 15: 3917-3917 | |
| | | (1987) | |
| | | JP1997322774 | |
| | | JP1993284970 | E14511 |
| | | Kim et al., Journal | E05776 |
| | | of Microbiology | D87976 |
| | | and Biotechnology | |
| | | 5: 250-256 (1995) | |
| Dps | Protection during starvation protein | EP1108790 | AX127153 |
| Eno | enolase | EP1108790 | AX127146 |
| | EC 4.2.1.11 | WO0100844 | AX064945 |
| | | EP1090998 | AX136862 |
| | | Hermann et al., | |
| | | Electrophoresis | |
| | | 19: 3217-3221 | |
| | | (1998) | |
| Gap | glyceraldehyde-3-phosphate | EP1108790 | AX127148 |
| | dehydrogenase | WO0100844 | AX064941 |
| | EC 1.2.1.12 | Eikmanns et al., | X59403 |
| | | Journal of | |
| | | Bacteriology | |
| | | 174: 6076-6086 | |
| | | (1992) | |
| gap2 | glyceraldehyde-3-phosphate | EP1108790 | AX127146 |
| | dehydrogenase 2 | WO0100844 | AX064939 |
| | EC 1.2.1.12 | | |
| Gdh | glutamate dehydrogenase | EP1108790 | AX127150 |
| | EC 1.4.1.4 | WO0100844 | AX063811 |
| | | Boermann et al., | X59404 |
| | | Molecular | |
| | | Microbiology | |
| | | 6: 317-326 (1992) | X72855 |
| Gnd | 6-phosphogluconate dehydrogenase | EP1108790 | AX127147 |
| | EC 1.1.1.44 | WO0100844 | AX121689 |
| | | | AX065125 |
| lysC | aspartate kinase | EP1108790 | AX120365 |
| | EC 2.7.2.4 | WO0100844 | AX063743 |
| | | Kalinowski et al., | X57226 |
| | | Molecular | |
| | | Microbiology | |
| | | 5: 1197-204 (1991) | |
| lysE | lysine exporter protein | EP1108790 | AX123539 |
| | | WO0100843 | AX123539 |
| | | Vrljic et al., | X96471 |
| | | Molecular | |
| | | Microbiology | |
| | | 22: 815-826 (1996) | |
| msiK | multiple sugar import protein | EP1108790 | AX120892 |
| opcA | subunit of glucose-6-phosphate | WO0104325 | AX076272 |
| | dehydrogenase | | |
| oxyR | transcriptional regulator | EP1108790 | AX122198 |
| | | | AX127149 |
| ppc^{FBR} | phosphoenolpyruvate carboxylase | EP1 196 611 | |
| | feedback resistant | WO0100852 | |
| | EC 4.1.1.31 | | |
| Ppc | phosphoenolpyruvate carboxylase | EP1108790 | AX127148 |
| | EC 4.1.2.31 | O'Reagan et al., | AX123554 |
| | | Gene 77(2): 237-251 | M25819 |
| | | (1989) | |
| Pgk | phosphoglycerate kinase | EP1108790 | AX121838 |
| | EC 2.7.2.3 | WO0100844 | AX127146 |
| | | Eikmanns, Journal | AX064943 |
| | | of Bacteriology | X59403 |
| | | 174: 6076-6086 | |
| | | (1992) | |
| pknA | protein kinase A | EP1108790 | AX120131 |
| | | | AX120085 |
| pknB | protein kinase B | EP1108790 | AX120130 |
| | | | AX120085 |
| pknD | protein kinase D | EP1108790 | AX127150 |
| | | | AX122469 |
| | | | AX122468 |
| pknG | protein kinase G | EP1108790 | AX127152 |
| | | | AX123109 |
| pptA | phosphoenolpyruvate synthase | EP1108790 | AX127144 |
| | EC 2.7.9.2 | | AX120700 |
| | | | AX122469 |
| ptsH | phosphotransferse system component H | EP1108790 | AX122210 |
| | EC 2.7.1.69 | WO0100844 | AX127149 |
| | | | AX069154 |
| ptsI | phosphotransferase system enzyme I | EP1108790 | AX122206 |
| | EC 2.7.3.9 | | AX127149 |
| ptsM | glucose-phosphotransferase-system | Lee et al., FEMS | L18874 |
| | enzyme II | Microbiology | |
| | EC 2.7.1.69 | Letters 119 (1-2): | |
| | | 137-145 (1994) | |
| Pyc | pyruvate carboxylase | WO9918228 | A97276 |
| | EC 6.4.1.1 | Peters-Wendisch et | Y09548 |
| | | al., Microbiology | |
| | | 144: 915-927 (1998) | |
| pyc | pyruvat-carboxylase | EP1108790 | |
| Pro458Ser | EC 6.4.1.1 | | |
| | aminoacid exchange Pro458Ser | | |
| sigC | extracytoplasmic function alternative | EP1108790 | AX120368 |
| | sigma factor C | | AX120085 |
| | EC 2.7.7.6 | | |
| sigD | RNA polymerase sigma factor D | EP1108790 | AX120753 |
| | EC 2.7.7.6 | | AX127144 |
| sigE | extracytoplasmic function alternative | EP1108790 | AX127146 |
| | sigma factor E | | AX121325 |
| | EC 2.7.7.6 | | |
| Sigh | sigma factor SigH | EP1108790 | AX127145 |
| | EC 2.7.7.6 | | AX120939 |
| sigM | sigma factor SigM | EP1108790 | AX123500 |
| | EC 2.7.7.6 | | AX127153 |
| Tal | transaldolase | WO0104325 | AX076272 |
| | EC 2.2.1.2 | | |
| thyA | thymidylate synthase | EP1108790 | AX121026 |
| | EC 2.1.1.45 | | AX127145 |
| Tkt | transketolase | Ikeda et al., | AB023377 |
| | EC 2.2.1.1 | NCBI | |
| Tpi | triose-phosphate isomerase | Eikmanns, Journal | X59403 |
| | EC 5.3.1.1 | of Bacteriology | |
| | | 174: 6076-6086 | |
| | | (1992) | |
| Zwal | growth factor 1 | EP1111062 | AX133781 |
| Zwf | glucose-6-phosphate-1-dehydrogenase | EP1108790 | AX127148 |
| | EC 1.1.1.49 | WO0104325 | AX121827 |
| | | | AX076272 |
| Ala213Thr | glucose-6-phosphate-1-dehydrogenase | EP1108790 | |
| (zwf | EC 1.1.1.49 | | |
| A213T) | Amino acid exchange 13T | | |

Furthermore, it can be advantageous for the production of L-lysine, in addition to amplification of one or more of the genes selected from the group consisting of amt, ocd, soxA and/or sumT, to simultaneously weaken one or more of the genes, selected from the group consisting of genes or alleles that are not essential for growth or for lysine production, particularly to reduce the expression.

This includes, among others, the following open read frames, genes, or alleles: aecD, ccpA1, ccpA2, citA, citB, citE, fda, gluA, gluB, gluC, gluD, luxR, luxS, lysR1, lysR2, lysR3, menE, mqo, pck, pgi, poxB, and zwa2, which are listed and explained in Table 2.

TABLE 2
Genes and alleles that are not essential for lysine production

| | Designation of | | |
|---|---|---|---|
| | the coded | | Access |
| Name | enzyme or protein | Reference | Number |
| aecD | beta C-S lyase | Rossol et al., Journal of | M89931 |
| | EC 2.6.11 | Bacteriology | |
| | | 174(9): 2968-77 (1992) | |
| ccpA1 | catabolite control | WO0100844 | AX065267 |
| | protein A1 | EP1108790 | AX127147 |
| | | WO 02/18419 | |
| ccpA2 | catabolite control | WO0100844 | AX065267 |
| | protein A2 | EP1108790 | AX121594 |
| citA | sensor kinase CitA | EP1108790 | AX120161 |
| citB | transcription regulator | EP1108790 | AX120163 |
| | CitB | | |
| citE | citrate lyase | WO0100844 | AX065421 |
| | EC 4.1.36 | EP1108790 | AX127146 |
| fda | fructose 1,6- | von der Osten et al., | X17313 |
| | bisphosphate aldolase | Journal of Bacteriology | |
| | EC 4.1.2.13 | 3(11): 1625-37(1989) | |
| gluA | glutamate transport | Kronemeyer et al., | X81191 |
| | ATP-binding protein | Journal of Bacteriology | |
| | | 177(5): 1152-8 (1995) | |
| gluB | glutamate binding | Kronemeyer et al., | X81191 |
| | system protein | Journal of Bacteriology | |
| | | 177(5): 1152-8 (1995) | |
| gluC | glutamate transport | Kronemeyer et al., | X81191 |
| | system permease | Journal of Bacteriology | |
| | | 177(5): 1152-8 (1995) | |
| gluD | glutamate transport | Kronemeyer et al., | X81191 |
| | system permease | Journal of Bacteriology | |
| | | 177(5): 1152-8 (1995) | |
| luxR | transcription regulator | WO0100842 | AX065953 |
| | LuxR | EP118790 | AX123320 |
| luxS | histidine kinase LuxS | EP1108790 | AX123323 |
| | | | AX127153 |
| lysR1 | transcription regulator | EP1108790 | AX064673 |
| | LysR1 | | AX127144 |
| lysR2 | transcription regulator | EP1108790 | AX123312 |
| | LysR2 | | |
| lysR3 | transcription regulator | WO0100842 | AX065957 |
| | LysR3 | EP118790 | AX127150 |
| menE | O-Succinylbenzoate- | WO0100843 | AX064599 |
| | CoA-ligase | EP1108790 | AX064193 |
| | EC 6.2.1.26 | | AX127144 |
| Mqo | malate-quinone- | Molenaar et al., Eur. | AJ224946 |
| | oxidoreductase | Journal of Biochemistry | |
| | | 1; 254(2): 395-403 | |
| | | (1998) | |
| pck | phosphoenolpyruvate | WO100844 | AJ269506 |
| | carboxykinase | EP-A-1094111 | AX065053 |
| pgi | glucose-6-phosphate | EP1087015 | AX136015 |
| | isomerase | EP1108790 | AX127146 |
| | EC 5.3.1.9 | WO 01/07626 | |
| poxB | pyruvate oxidase | WO0100844 | AX064959 |
| | EC 1.2.3.3 | EP1096013 | AX137665 |
| zwa2 | growth factor 2 | EP1106693 | AX113822 |
| | | EP1108790 | AX127146 |

Finally, it can be advantageous for the production of amino acids, in addition to amplification of one or more of the genes selected from the group consisting of amt, ocd, soxA and/or sumT, to eliminate undesirable secondary reactions (Nakayama: “Breeding of Amino Acid Producing Micro-organisms,” in: Overproduction of Microbial Products, Krumphanzl, Sikyta, Vanek (eds.), Academic Press, London, UK 1982)).

The microorganisms produced according to the present invention can be cultivated continuously or discontinuously, using the batch method, or the fed batch method or the repeated fed batch method, for the purpose of the production of L-amino acids. A summary of known cultivation methods is described in the textbook by Chmiel (Bioprozesstechnik 1. Einführung in die Bioverfahrenstechnik [Bioprocess Technology 1. Introduction to Bioprocess Technology] (Gustav Fischer Verlag, Stuttgart, 1991) or in the textbook by Storhas (Bioreaktoren und periphere Einrichtungen [Bioreactors and Peripheral Equipment] (Vieweg Verlag, Braunschweig/Wiesbaden, 1994).

The culture medium to be used must satisfy the requirements of the strains, in each instance, in a suitable manner. Descriptions of culture media for various microorganisms are contained in the manual “Manual of Methods for General Bacteriology” of the American Society for Bacteriology (Washington, D.C., USA, 1981).

Sugar and carbohydrates such as glucose, saccharose, lactose, fructose, maltose, molasses, starch and cellulose, for example, oils and fats such as soybean oil, sunflower oil, peanut oil, and coconut oil, for example, fatty acids such as palmitic acid, stearic acid, and linoleic acid, for example, alcohols such as glycerin and ethanol, for example, and organic acids such as acetic acid, for example, can be used as carbon sources. These substances can be used individually or in mixtures.

Organic compounds that contain nitrogen, such as peptones, yeast extract, meat extract, malt extract, corn source water, soybean oil, and urea, or inorganic compounds such as ammonium sulfate, ammonium chloride, ammonium phosphate, ammonium carbonate, and ammonium nitrate can be used as nitrogen sources. The nitrogen sources can be used individually or as mixtures.

Phosphoric acid, potassium dihydrogen phosphate, or dipotassium hydrogen phosphate, or the corresponding salts containing sodium, can be used as phosphorus sources. Furthermore, the culture medium must contain salts of metal such as magnesium sulfate or iron sulfate, for example, which are necessary for the medium. Finally, essential growth substances such as amino acids and vitamins can be used, in addition to the aforementioned substances. In addition, suitable precursor stages can be added to the culture medium. The said substances for use can be added to the culture in the form of a one-time batch, or be fed in during cultivation, in suitable manner.

In order to control the pH of the culture, basic compounds such as sodium hydroxide, potassium hydroxide, ammonia or water of ammonia, or acidic compounds such as phosphoric acid or sulfuric acid, are used in suitable manner. To control foam development, anti-foam agents such as fatty acid polyglycol ester, for example, can be used. To maintain the stability of plasmids, suitable substances having a selective effect, such as antibiotics, for example, can be added to the medium. In order to maintain aerobic conditions, oxygen or gas mixtures containing oxygen, such as air, for example, are supplied to the culture. The temperature of the culture normally lies between 20° C. and 45° C., and preferably between 25° C. and 40° C. The temperature of the culture includes all values and subvalues therebetween, especially including 22, 24, 26, 28, 30, 32, 34, 36, 38, 40, 42 and 44° C. Culturing is continued until a maximum of the desired product has formed. This goal is normally achieved within 10 hours to 160 hours. The time to obtain a maximum of the desired product includes all values and subvalues therebetween, especially including 20, 30, 40, 50, 60, 70, 80, 90, 100, 110, 120, 130, 140 and 150 hours.

Using the methods of the present invention, the output of the bacteria or of the fermentation process, with regard to the product concentration (product per volume), the product yield (product formed per carbon source used up), the product formation (product formed per volume and time), or other process parameters or combinations of them, can be improved by at least 0.5%, preferably at least 1%, and more preferably at least 2%.

Methods for the determination of L-amino acids are known from the state of the art. The analysis can take place as described by Spackman et al. (Analytical Chemistry, 30, (1958), 1190), by means of anion exchange chromatography, with subsequent ninhydrin derivation, or it can take place by means of reversed-phase HPLC, as described by Lindroth et al. (Analytical Chemistry (1979) 51: 1167-1174).

The following figures are attached:

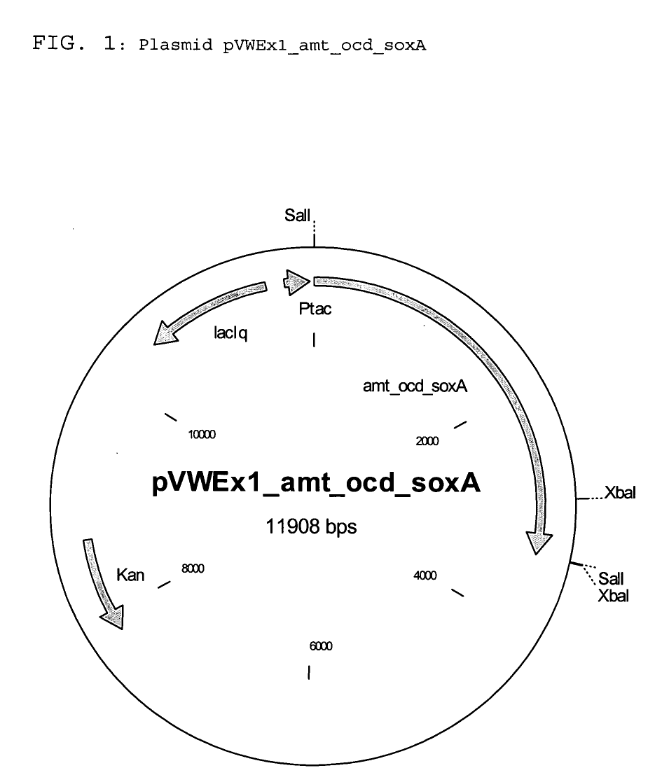

FIG. 1: Map of the plasmid pVWEx1_amt_ocd_soxA.

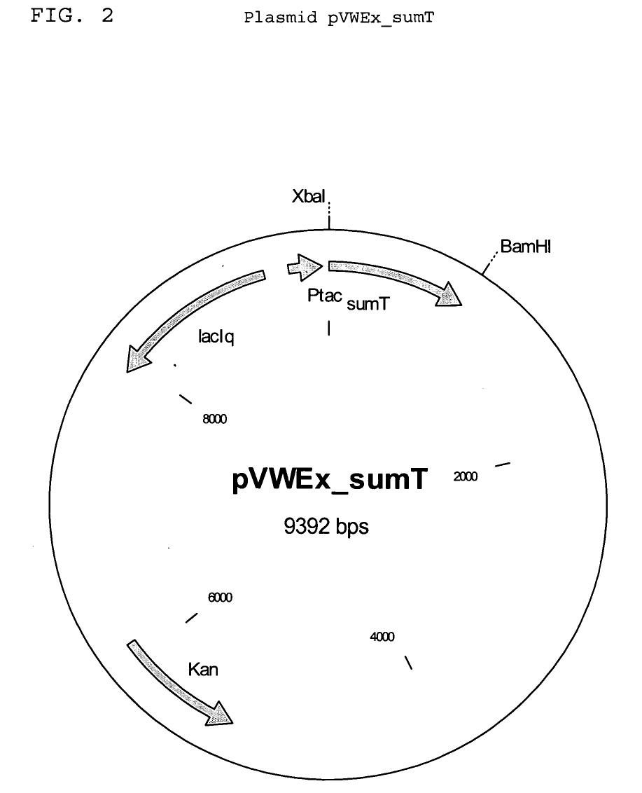

FIG. 2: Map of the plasmid pVWEx1_sumT.

The data relating to the base pair numbers involve approximation values that are obtained within the framework of the reproducibility of measurements.

The abbreviations and designations used have the following meanings:

| | |
|---|---|
| amt_ocd_{—} | PCR fragment having ribosome binding points and amt, |
| soxA: | ocd, and soxA, |
| sumT: | PCT fragment having ribosome binding point and sumT, |
| Km: | kanamycin resistance gene, |
| lacIq: | lac repressor gene lacIQ, |
| Ptac: | tac promoter, |
| SalI: | cutting point of the restriction enzyme SalI, |
| XbaI: | cutting point of the restriction enzyme XbaI, and |
| BamHI: | cutting point of the restriction enzyme BamHI. |

Having generally described this invention, a further understanding can be obtained by reference to certain specific examples which are provided herein for purposes of illustration only, and are not intended to be limiting unless otherwise specified.

### EXAMPLES

### Example 1

### Production of the Shuttle Vector pVWEx1-amt_ocd_soxA for Amplification of the Genes amt, ocd, and soxA in C. glutamicum

### 1.1 Cloning of the Genes amt, ocd, and soxA

Chromosomal DNA was isolated from the strain ATCC 13032, according to the method of Eikmanns et al. (Microbiology 140: 1817-1828 (1994)). On the basis of the sequence of the genes amt, ocd, and soxA known for C. glutamicum, the following oligonucleotides were selected for the polymerase chain reaction. In addition, a suitable ribosome binding point and suitable restriction cutting points were inserted, which allowed cloning into the target vector:

(SEQ ID No. 1)

amt_ocd_soxA-frw
5′ AC GC GTCGAC AAGGAGAAGGGC C ATG GAC CCC TCA GAT

CTA G 3′
(SEQ ID No. 2)

amt_ocd_soxA-rev
5′ ACGC GTCGA CAC CGA GGG CAC ATC GGT G 3′

The primers shown were synthesized by MWG (Ebersbach, Germany). The primer amt_ocd_soxA-frw contained the sequence for the cutting point of the restriction endonuclease Sal1, and the primer amt_ocd_soxA-rev contained the cutting point of the restriction endonuclease Sal1, which are marked by underlining in the nucleotide sequence shown above. The PCR reaction was carried out according to the standard PCR method of Innis et al. (PCR protocols. A guide to methods and applications, 1990, Academic Press), using a mixture of Taq and Tgo polymerase from Roche Diagnostics GmbH (Mannheim, Germany). Using the polymerase chain reaction, the primers allow amplification of a DNA fragment having a size of 3393 bp, which carried the genes amt, ocd, and soxA from Corynebacterium glutamicum, without a potential promoter region, but with an inserted ribosome binding point (SEQ ID No. 3). The fragment amplified in this manner was checked by electrophoresis in a 1% agarose gel.

The PCR fragment obtained in this manner was completely split using the restriction enzyme Sal1 and, after separation, was isolated from the gel in a 1% agarose gel, using the QiaExII Gel Extraction Kit (Product No. 20021, Qiagen, Hilden, Germany).

### 1.2 Cloning of the Genes amt, ocd, and soxA into the Vector pVWEx1

The E. coli-C. glutamicum-shuttle-expression vector pVWEx1 (Peters-Wendisch et al., Journal of Molecular Microbiology and Biotechnology 3(2): 295-300 (2001)) was used as the base vector for the expression both in C. glutamicum and in E. coli. DNA of this plasmid was completely split using the restriction enzyme Sal1, and subsequently dephosphorylated using shrimp alkaline phosphatase (Roche Diagnostics GmbH, Mannheim, Germany, product description SAP, Product No. 1758250). The fragment isolated from the agarose gel in Example 1.1, which carried the genes amt, ocd, soxA, as well as a ribosome binding point, was mixed with the vector pVWEx1 prepared in this manner, and the batch was treated with T4-DNA-ligase (Amersham Pharmacia, Freiburg, Germany).

The ligation batch was transformed into the E. coli strain DH5αmcr (Hanahan, in: DNA Cloning. A Practical Approach. Vol. I. IRL-Press, Oxford, Washington D.C., USA). The selection of plasmid-carrying cells took place by means of unplating of the transformation batch onto LB agar (Lennox, 1955, Virology, 1:190), with 50 mg/l kanamycin. After incubation overnight, at 37° C., recombinant individual clones were selected. Plasmid DNA was isolated from a transformant, using the Qiaprep Spin Miniprep Kit (Product No. 27106, Qiagen, Hilden, Germany), according to the manufacturer's instructions, and checked by means of restriction splitting. The plasmid obtained was called pVWEx1-amt_ocd_soxA. It is shown in FIG. 1.

### Example 2

### Transformation of the Strain DSM5715 Using the Plasmid pVWEx1-amt_ocd_soxA

In order to demonstrate the superiority of the method claimed, experiments were conducted with the L-lysine-producing strain Corynebacterium glutamicum DSM5715 (EP-B-0 435 132). This strain was developed by means of mutagenesis of Corynebacterium glutamicum ATCC13032, with nitronitrosoguanidine, and contained a feedback-resistant aspartate kinase.

The strain DSM5715 was transformed using the plasmid pVWEx1-amt_ocd_soxA, using the electroporation method described by Liebl et al. (FEMS Microbiology Letters, 53: 299-303 (1989)). The selection of the transformants took place on LBHIS agar, consisting of 18.5 g/l brain-heart infusion bouillon, 0.5 M sorbitol, 5 g/l bacto-tryptone, 2.5 g/l bacto-yeast extract, 5 g/l NaCl, and 18 g/l bacto-agar, which was supplemented with 25 mg/l kanamycin. The incubation took place for 2 days at 30° C.

Plasmid DNA was isolated from a transformant, using the usual methods (Peters-Wendisch et al., 1998, Microbiology, 144, 915-927), cut with the restriction enzyme Sal1, and the plasmid was checked by means of subsequent agarose gel electrophoresis. The strain obtained was called DSM5715/pVWEx1-amt_ocd_soxA.

### Example 3

### Production of Lysine

The C. glutamicum strain DSM5715/pVWEx1-amt_ocd_soxA obtained in Example 2 was cultivated in a nutrient medium suitable for the production of lysine, and the lysine content in the top fraction of the culture was determined.

For this purpose, the strain was first incubated on an agar plate, with the corresponding antibiotic (LB agar with kanamycin (25 mg/l)) for 24 hours at 30° C. Proceeding from this agar plate culture, a pre-culture was inoculated (5 ml medium in 10 ml test tube). The full medium CgIII was used for the pre-culture.

Medium Cg III

| | | |
|---|---|---|
| NaCl | 2.5 | g/l |
| Bacto-peptone | 10 | g/l |
| Bacto-yeast extract | 10 | g/l |
| Glucose (autoclaved separately) | 2% | (w/v) |
| The pH is adjusted to pH 7.4. | | |

Kanamycin (25 mg/l) was added to this. The pre-culture was incubated on a shaker for 16 hours at 30° C. and 180 rpm. A second pre-culture was inoculated from this pre-culture (50 ml medium in a 500 ml Erlenmeyer flask) and incubated on a shaker for 24 h at 30° C. and 240 rpm. The minimal medium CgXII, with 10% (w/v) glucose, to which kanamycin (25 mg/l) was added, was used as the medium for the second pre-culture.

A main culture was inoculated from this second pre-culture, so that the starting OD (600 nm) of the main culture is 0.5 OD. The medium CGXII was used for the main culture.

Medium CGXII

| | | |
|---|---|---|
| Urea | 5 | g/l |
| MOPS (morpholinopropane sulfonic acid) | 42 | g/l |
| Glucose (autoclaved separately) | 100 | g/l |
| Salts: | | |
| (NH₄)₂SO₄ | 20 | g/l |
| KH₂PO₄ | 1.0 | g/l |
| K₂HPO₄ | 1.0 | g/l |
| MgSO₄ * 7 H₂O | 0.25 | g/l |
| CaCl₂ * 2 H₂O | 10 | mg/l |
| FeSO₄ * 7 H₂O | 10 | mg/l |
| MnSO₄ * H₂O | 10 | mg/l |
| ZnSO₄ * 7 H₂O | 1.0 | mg/l |
| CuSO | 0.2 | mg/l |
| NiCl₂ * 6 H₂O | 0.02 | mg/l |
| Biotin (sterile-filtered) | 0.2 | mg/l |
| Protekatechuate (sterile-filtered) | 0.03 | mg/l |
| Leucine (sterile-filtered) | 0.1 | g/l |

Urea, MOPS, and the salt solution were adjusted to pH 7 with KOH, and autoclaved. The glucose solution was autoclaved separately. Subsequently, the sterile substrate solution, amino acid solution, and vitamin solution were added.

Cultivation took place in 50 ml volume in a 500 ml Erlenmeyer flask with baffles. Kanamycin (25 mg/l) was added. Cultivation took place at 30° C. and 85% relative humidity.

After 72 hours, the OD was determined at a measurement wavelength of 600 nm, using the Biomek 1000 (Beckmann Instruments GmbH, Munich). The amount of lysine formed was determined by means of HPLC (liquid chromatography), using a Hewlett-Packard HPLC device Type HP1100, and o-phthaldialdehyde derivation using a fluorescence detector G1321A (Jones & Gilligan 1983).

The results of the experiment are shown in Table 3.

TABLE 3

| Strain | OD (600) | Lysine HCl mM |
|---|---|---|
| DSM5715 | 30 | 76 |
| DSM5715/pVWEx1- | 30 | 125 |
| amt_ocd_soxA | | |

### Example 4

### Production of the Shuttle Vector pVWEx1-sumT for Amplification of the sumT Gene in C. glutamicum

### 4.1 Cloning of the sumT Gene

Chromosomal DNA was isolated from the strain ATCC 13032, according to the method of Eikmanns et al. (Microbiology 140: 1817-1828 (1994)). On the basis of the sequence of the sumT gene known for C. glutamicum, the following oligonucleotides were selected for the polymerase chain reaction. In addition, a suitable ribosome binding point and suitable restriction cutting points were inserted, which allowed cloning into the target vector:

(SEQ ID No. 4)

sumT-frw
5′ GC TCTAG AAGGAGATTCTCC ATG CAT GTT GCT GAA TTA

TC 3′
(SEQ ID No. 5)

sumTneu-rev
5′ CG GGATC CGAT TAA TTT TCC CTG GCA G 3′

The primers shown were synthesized by MWG (Ebersberg, Germany). The primer sumT-frw contained the sequence for the cutting point of the restriction endonuclease Xba1, and the primer sumTneu-rev contained the cutting point of the restriction endonuclease BamH1, which were marked by underlining in the nucleotide sequence shown above. The PCR reaction was carried out according to the standard PCR method of Innis et al. (PCR protocols. A guide to methods and applications, 1990, Academic Press), using a mixture of Taq and Tgo polymerase from Roche Diagnostics GmbH (Mannheim, Germany). Using the polymerase chain reaction, the primers allow amplification of a DNA fragment having a size of 879 bp, which carried the sumT gene from Corynebacterium glutamicum, without a potential promoter region (SEQ ID No. 6). The fragment amplified in this manner was checked by electrophoresis in a 1% agarose gel.

The PCR fragment obtained in this manner was completely split using the restriction enzymes Xba1 and BamH1 and, after separation, was isolated from the gel in a 1% agarose gel, using the QiaEXII Gel Extraction Kit (Product No. 20021, Qiagen, Hilden, Germany).

### 4.2 Cloning of sumT Gene into the Vector pVWEx1

The E. coli-C. glutamicum-shuttle-expression vector pVWEx1 (Peters-Wendisch et al., 2001) was used as the base vector for the expression both in C. glutamicum and in E. coli. DNA of this plasmid was completely split using the restriction enzymes Xba1 and BamH1, and subsequently dephosphorylated using shrimp alkaline phosphatase (Roche Diagnostics GmbH, Mannheim, Germany, product description SAP, Product No. 1758250). The sumT fragment isolated from the agarose gel in Example 4.1 was mixed with the vector pVWEx1 prepared in this manner, and the batch was treated with T4-DNA-ligase (Amersham Pharmacia, Freiburg, Germany).

The ligation batch was transformed into the E. coli strain DH5αmcr (Hanahan, in: DNA Cloning. A Practical Approach. Vol. I. IRL-Press, Oxford, Washington D.C., USA). The selection of plasmid-carrying cells took place by means of unplating of the transformation batch onto LB agar (Lennox, 1955, Virology, 1:190), with 50 mg/l kanamycin. After incubation overnight, at 37° C., recombinant individual clones were selected. Plasmid DNA was isolated from a transformant, using the Qiaprep Spin Miniprep Kit (Product No. 27106, Qiagen, Hilden, Germany), according to the manufacturer's instructions, and checked by means of restriction splitting. The plasmid obtained was called pVWEx1-sumT. It is shown in FIG. 2.

### Example 5

### Transformation of the Strain DSM5715 Using the Plasmid pVWEx1-sumT

The strain DSM5715 was transformed using the plasmid pVWEx1-sumT, using the electroporation method described by Liebl et al. (FEMS Microbiology Letters, 53: 299-303 (1989)). The selection of the transformants took place on LBHIS agar, consisting of 18.5 g/l brain-heart infusion bouillon, 0.5 M sorbitol, 5 g/l bacto-tryptone, 2.5 g/l bacto-yeast extract, 5 g/l NaCl, and 18 g/l bacto-agar, which was supplemented with 25 mg/l kanamycin. The incubation took place for 2 days at 30° C.

Plasmid DNA was isolated from a transformant, using the usual methods (Peters-Wendisch et al., 1998, Microbiology, 144, 915-927), cut with the restriction endonucleases Xba1 and BamH1, and the plasmid was checked by means of subsequent agarose gel electrophoresis.

The strain obtained was called DSM5715/pVWEx1-sumT.

### Example 6

### Production of Lysine

The C. glutamicum strain DSM5715//pVWEx1-sumT obtained in Example 5 was cultivated in a nutrient medium suitable for the production of lysine, and the lysine content in the top fraction of the culture was determined.

For this purpose, the strain was first incubated on an agar plate, with the corresponding antibiotic (LB agar with kanamycin (25 mg/l)) for 24 hours at 30° C. Proceeding from this agar plate culture, a pre-culture was inoculated (5 ml medium in 10 ml test tube). The full medium CgIII was used for the pre-culture.

Medium Cg III

| | | |
|---|---|---|
| NaCl | 2.5 | g/l |
| Bacto-peptone | 10 | g/l |
| Bacto-yeast extract | 10 | g/l |
| Glucose (autoclaved separately) | 2% | (w/v) |
| The pH is adjusted to pH 7.4. | | |

Kanamycin (25 mg/l) was added to this. The pre-culture was incubated on a shaker for 16 hours at 30° C. and 180 rpm. A second pre-culture was inoculated from this pre-culture (50 ml medium in a 500 ml Erlenmeyer flask) and incubated on a shaker for 24 h at 30° C. and 240 rpm. The minimal medium CgXII, with 10% (w/v) glucose, to which kanamycin (25 mg/l) was added, was used as the medium for the second pre-culture.

A main culture was inoculated from this second pre-culture, so that the starting OD (600 nm) of the main culture is 0.5 OD. The medium CGXII was used for the main culture.

Medium CGXII

| | | |
|---|---|---|
| Urea | 5 | g/l |
| MOPS (morpholinopropane sulfonic acid) | 42 | g/l |
| Glucose (autoclaved separately) | 100 | g/l |
| Salts: | | |
| (NH₄)₂SO₄ | 20 | g/l |
| KH₂PO₄ | 1.0 | g/l |
| K₂HPO₄ | 1.0 | g/l |
| MgSO₄ * 7 H₂O | 0.25 | g/l |
| CaCl₂ * 2 H₂O | 10 | mg/l |
| FeSO₄ * 7 H₂O | 10 | mg/l |
| MnSO₄ * H₂O | 10 | mg/l |
| ZnSO₄ * 7 H₂O | 1.0 | mg/l |
| CuSO | 0.2 | mg/l |
| NiCl₂ * 6 H₂O | 0.02 | mg/l |
| Biotin (sterile-filtered) | 0.2 | mg/l |
| Protekatechuate (sterile-filtered) | 0.03 | mg/l |
| Leucine (sterile-filtered) | 0.1 | g/l |

Urea, MOPS, and the salt solution were adjusted to pH 7 with KOH, and autoclaved. The glucose solution was autoclaved separately. Subsequently, the sterile substrate solution, amino acid solution, and vitamin solution were added.

Cultivation took place in 50 ml volume in a 500 ml Erlenmeyer flask with baffles. Kanamycin (25 mg/l) was added. Cultivation took place at 30° C. and 85% relative humidity.

After 72 hours, the OD was determined at a measurement wavelength of 600 nm, using the Biomek 100 (Beckmann Instruments GmbH, Munich). The amount of lysine formed was determined by means of HPLC (liquid chromatography), using a Hewlett-Packard HPLC device Type HP1100, and o-phthaldialdehyde derivation using a fluorescence detector G1321A (Jones & Gilligan 1983).

The results of the experiment are shown in Table 4.

TABLE 4

| Strain | OD (600) | Lysine HCl mM |
|---|---|---|
| DSM5715 | 30 | 76 |
| DSM5715/pVWEx1-sumT | 30 | 128 |

German patent application 10344739.3 filed Sep. 26, 2003, and all patents and references mentioned in the specification are incorporated herein by reference.

Numerous modifications and variations on the present invention are possible in light of the above teachings. It is therefore to be understood that within the scope of the appended claims, the invention may be practiced otherwise than as specifically described herein.

1. A method for producing an L-amino acid, comprising:
fermenting a medium using coryneform bacteria in which one or more of the genes linked with a nitrogen metabolism and selected from the group consisting of amt, ocd, soxA and sumT is/are amplified.

2. The method according to claim 1, wherein a concentration of the proteins coded by the said genes is increased by 10 to 2000%.

3. The method according to claim 1, wherein L-lysine is produced.

4. A method for producing an L-amino acid, comprising:
a) fermenting a medium using recombinant coryneform bacteria that produce said L-amino acid,
wherein in said bacteria at least one or more of the genes selected from the group consisting of amt, ocd, soxA and sumT is/are amplified;
b) accumulating said L-amino acid in said medium or in the cells of said bacteria, and
c) isolating said L-amino acid.

5. The method according to claim 1, wherein, in said bacteria, additionally other genes of a biosynthesis path of said L-amino acid are amplified.

6. The method according to claim 1, wherein, in said bacteria, metabolic paths that reduce the formation of said L-amino acid are at least partially shut off.

7. The method according to claim 1, wherein at least one polynucleotide that codes for one or more of the genes selected from the group consisting of amt, ocd, soxA and sumT is over-expressed.

8. The method according to claim 1, wherein at least one regulatory and/or catalytic property of at least one polypeptide, for which the polynucleotides selected from the group consisting of amt, ocd, soxA and sumT code, is amplified.

9. The method according to claim 5, wherein a concentration of at least one protein for which said amplified gene codes is increased by 10 to 2000%.

10. The method according to claim 5, wherein said bacteria are coryneform bacteria in which one or more of the genes selected from the group consisting of accBC, accDA, cstA, cysD, cysE, cysH, cysK, cysN, cysQ, dapA, dapB, dapC, dapD, dapE, dapF, ddh, dps, eno, gap, gap2, gdh, gnd, lysC, lysCFBR, lysE, msiK, opcA, oxyR, ppc, ppc^{FBR}, pgk, pknA, pknB, pknD, pknG, ppsA, ptsH, ptsI, ptsM, pyc, pyc Pro458Ser, sigc, sigD, sigE, sigH, sigM, ta1, thyA, tkt, tpi, zwa1, zwf, and Ala213Thr is/are amplified.

11. The method according to claim 6, wherein an activity and/or a concentration of the protein(s) for which the weakened gene(s) code(s) drops to 0 to 75%, in each instance.

12. The method according to claim 6, wherein, in said bacteria, one or more of the genes selected from the group consisting of aecD, ccpA1, ccpA2, citA, citB, citE, fda, gluA, gluB, gluC, gluD, luxR, luxS, lysR1, lysR2, lysR3, menE, mqo, pck, pgi, poxB, and zwa2 is/are weakened, shut off, or have a reduced expression.

13. The method according to claim 1, wherein said bacteria are of the species Corynebacterium glutamicum.

14. Coryneform bacteria in which at least one or more of the genes selected from the group consisting of amt, ocd, soxA and sumT is/are present in amplified form.

15. The method according to claim 1, wherein said bacteria are recombinant bacteria.

16. The method according to claim 1, wherein at least two of said genes are amplified.

17. The method according to claim 1, wherein at least one of said genes is over-expressed.

18. The method according to claim 1, wherein at least two of said genes are over-expressed.

19. The method according to claim 1, wherein an activity of the proteins coded by the said genes is increased by 10 to 2000%.

20. The method according to claim 4, wherein L-lysine is produced.

21. The method according to claim 4, wherein at least two of said genes are amplified.

22. The method according to claim 4, wherein at least one of said genes is over-expressed.

23. The method according to claim 4, wherein at least two of said genes are over-expressed.

24. The method according to claim 4, wherein said medium comprises a fermentation liquid and a biomass, and
wherein after said isolation of said L-amino acid, at least one component of the fermentation liquid and/or biomass remains in said L-amino acid, in its entirety or in a portion of from >0 to <100%.

25. The method according to claim 5, wherein said other genes are over-expressed.

26. The method according to claim 5, wherein an activity of at least one protein for which said amplified gene codes is increased by 10 to 2000%.

27. The method according to claim 10, wherein at least one of said genes is over-expressed.

28. The coryneform bacteria according to claim 27 in which at least one or more of the genes selected from the group consisting of amt, ocd, soxA and sumT is/are present in over-expressed form.

29. The method according to claim 4, wherein, in said bacteria, additionally other genes of a biosynthesis path of said L-amino acid are amplified.

30. The method according to claim 4, wherein, in said bacteria, metabolic paths that reduce the formation of said L-amino acid are at least partially shut off.

31. The method according to claim 4, wherein at least one polynucleotide that codes for one or more of the genes selected from the group consisting of amt, ocd, soxA and sumT is over-expressed.

32. The method according to claim 4, wherein at least one regulatory and/or catalytic property of at least one polypeptide, for which the polynucleotides selected from the group consisting of amt, ocd, soxA and sumT code, is amplified.

33. The method according to claim 29, wherein a concentration of at least one protein for which said amplified gene codes is increased by 10 to 2000%.

34. The method according to claim 29, wherein said bacteria are coryneform bacteria in which one or more of the genes selected from the group consisting of accBC, accDA, cstA, cysD, cysE, cysH, cysK, cysN, cysQ, dapA, dapB, dapC, dapD, dapE, dapF, ddh, dps, eno, gap, gap2, gdh, gnd, lysC, lysCFBR, lysE, msiK, opcA, oxyR, ppc, ppc^{FBR}, pgk, pknA, pknB, pknD, pknG, ppsA, ptsH, ptsI, ptsM, pyc, pyc Pro458Ser, sigc, sigD, sigE, sigH, sigM, ta1, thyA, tkt, tpi, zwa1, zwf, and Ala213Thr is/are amplified.

35. The method according to claim 30, wherein an activity and/or a concentration of the protein(s) for which the weakened gene(s) code(s) drops to 0 to 75%, in each instance.

36. The method according to claim 30, wherein, in said bacteria, one or more of the genes selected from the group consisting of aecD, ccpA1, ccpA2, citA, citB, citE, fda, gluA, gluB, gluC, gluD, luxR, luxS, lysR1, lysR2, lysR3, mene, mqo, pck, pgi, poxB, and zwa2 is/are weakened, shut off, or have a reduced expression.

37. The method according to claim 4, wherein said bacteria are of the species Corynebacterium glutamicum.


SEQUENCE LISTING (6 sequences)

SEQ ID NO: 1 - DNA, 42 bp, Artificial Sequence
  Synthetic DNA
acgcgtcgacaaggagaagggccatggacccctcagatctag

SEQ ID NO: 2 - DNA, 28 bp, Artificial Sequence
  Synthetic DNA
acgcgtcgacaccgagggcacatcggtg

SEQ ID NO: 3 - DNA, 3392 bp, Artificial Sequence
  Synthetic DNA
acgcgtcgacaaggagaagggccatggacccctcagatctagcctggattctcgcagcttttgcgttggtaagcctgatgttccccggattgtccctgctctacggcggcatgctgggtgggcaacacgttcttaacacgttcatgatggttatgagctcacttggaatcatcagccttgtgtacatcatttatggacacggacttgtcttaggaaactccatcggtgggtggggaattatcggaaatccccttgaatacttcggcttccgcaacattatggaagatgacggcaccggagacctcatgtgggccggcttctacattctgttcgctgcaatctcactcgcacttgtttcatctggtgcagcggggcgcatgcgctttggagcgtggctggtcttcggtgtcctgtggttcacctttgtgtacgcgccactggcacactgggttttcgctatcgatgatcctgagtccggctacgtgggtggctggatgaaaaatgtgcttgagttccacgactttgctggtggaacggcagtgcacatgaatgcgggtgcgtctggactcgcgctggcaatagtgctgggacgccgccactccatggctgtgcgtccacacaaccttccactgattttgattggtgcaggactgatcgttgcgggctggttcggattcaatggtggtaccgcaggtggtgccaacttcctcgcaagctacgtggtcgttacctctctcattgctgcagctggcggaatgatgggcttcatgctcgttgaacgtgtgttcagcggaaaacccactttctttggctcggcaaccggcacaatcgcaggccttgtggctatcaccccggccgcggatgcagtgagcccgctcggagcattcgccgtcggagcgctcggcgcagttgtctccttctgggcaattagctggaagaagggacaccgagtcgatgattccttcgatgtgttcgcagtccacggaatggccggcattgcaggtgcactgtttgtcatgctctttggcgatccactagcaccagcgggagtttccggagtcttcttcggtggcgaactctccctgctgtggagggaaccactggccatcatcgtgacccttacatacgcattcggcgtgacctggttgattgccacgatcttgaacaagttcatgactctgcgcatcacctccgaagccgaatatgaaggcattgaccgcgcagaacacgcagaatctgcctaccacctcaattccaacggaattgggatggcaacccgcaccaatttcggacctgaaatccccgaggaaaccgtgcccgacgccgtgcaggtgggcgtcgataagcaaaaaatcgctgatactcgaaaggcctcaaaatgaccgcaacctacaccactgaaaccgccatcaatttcttgttcttgagcgaaccggacatgatcgcggccggagtcaaagacgtcgcgcaatgcgtcgatgtcatggaggaaacgctcgtgctcttggcgcagggcgactacaaaatggccggtttgaactccaactcgcatggcgcgatgatcaccttcccggaaaacccagaatttgaaggcatgcccaaggacggccccgaccgccgattcatggcgatgcccgcatacctcggcgggcgattcaaaaacaccggcgtgaagtggtacggatccaacgcggaaaacaaggcctcaggcttgcctcgctcgatccacaccttcgtcctcaacgacacggtcaccggtgcaccgaaggccatcatgtccgcgaacctgctgtccgcctaccgcaccggcgcggttcccggcgtgggcgtgaagcacttagcggtcgccgacgcgacaaccttggctgtcgtcggacctggtgtcatggcgaaaaccatcaccgaagcgtgcatcgcagagcgcccaggaatcaccaccatcaagatcaagggacgcagcgaacgcggcatcaacgcctttgcaacatgggcgttggaaaaattccccgagatcgaagtggtcgccgtcggatctgaagaagacgtggtcaaagacgccgacatcgtcatcgccgccaccaccacggacgccgccggctcctccgccttcccatacttcaaaaaagaatggctcaagccgggcgcattgctgctgcttccagccgccggtcgcttcgacgacgcttatttgcttgacgacgcccgcctcgttgttgactacatggggctctacgaagcctgggcagaagaatacggcccacaggcctaccaactactcggcattccaggaacccactggtacgacctggcgctgcaaggaaaactcgaccttgcaaagatttcccagattggcgatatctgctccggcaagctacccggacgcaccaacgatgaggaaatcatcctctattccgtcggcggcatgccagtagaagacgtcgcctgggcaacccaagtgtatgaaaacgccctggaaaaaggcgtcggcaccacattgaacctgtgggaatcacccgcactggcttgagagaagaaacaacaatgaaaattgcggtaatcggccttggatcaaccggctccatggcactgtggcacttaagtaacatcccaggtgtagaggccatcggctttgaacaattcggcatctcccatggctacggcgcattcacaggggagtcccgactgtttcgcatggcctaccacgaaggcagcacctacgttccgttgctcaaacgcgcacgagcactatggtcatcactgagcgagatttccggacgcgaactcttccacaacttcggtgtcttaagcaccggcaaggaagacgaagcacccttccaacgcctggtggaatcagtggaacgttatgagctgccacatgaacgacttaccgccgcgcagatgcgcaagcgttacccaggtctagacttccgcgatgatgaagctggaattgttgatcttcaaggtggagccctgcgccccgaactagcagtgttcagtgcaatcgaaacagccaaggcaaatggtgcccaagtacgcgatcaccaaaaaatcaccagcatcgaagacaacggcgatcacgtagtcatccaagcaggcgaagaaaccacaatcgtggaccgcgttatcgtcaccaccggcagctggacaagcgagctcgtgccctccatcgcgccactgcttgaagtgcgacgcctagtgctcacctggttcctgcccaacaatccagtggacttccaaccggaaaacctgccatgcttcatccgtgaccgtgatggcttccacgtatttggagcaccatgcgtcgatgggtacagcatcaaaattgccggattggatgagtggggcgttccattaagcctcgatccaccgatgtgccctcggtgtcgacgcgt

SEQ ID NO: 4 - DNA, 40 bp, Artificial Sequence
  Synthetic DNA
gctctagaaggagattctccatgcatgttgctgaattatc

SEQ ID NO: 5 - DNA, 27 bp, Artificial Sequence
  Synthetic DNA
cgggatccgattaattttccctggcag

SEQ ID NO: 6 - DNA, 879 bp, Artificial Sequence
  Synthetic DNA
gctctagaaggagattctccatgcatgttgctgaattatctttgcccactggaattattatcgcggcgactccgctcggcaacattggggatgcgtctccgcgcctggtccacgcgcttgccaacgccactgtggtagctgcggaggatacccgcaggacggcgtccttggctgctgcgttgggggtggaaattaaggggcagttggtctcgaactttgaccataatgaacaggcgcgcgtcggcaagcttattgaagcagcgcgcacgggcacggtgctggtggtcagcgatgccggcatgcctgtggtttctgatccgggttttgcgcttatcgacgccgcccacgacgcgaacattccggtcacctgcttccccgggccgtcagctgtgccaactgcgttggcattgtcgggccttcacgtgggccgctttgccttcgacggtttcgcgccgcgcaaacaaggtgcgcgcaccacgtggttggagtcgttgaaaaccgaaaagcgcgcggtatgtttcttcgaatctcctcaccgcatcgcagaaaccctggctcacgctgccgaagttttaggtgaacgacgcgtagcagtgtgccgtgaactgtccaaaacctacgaacaggtaaagcgtggaaccttgccagagttggcagaatgggcacaagatggggtgcgtggcgagatcaccgttgtcatcgaaggcgcgggcgatatcgcggccgacgtcgattcgcttatcgacgccgcccagcagcgcgtcgattccggcgagcggttgaaagcggtgtgcgcagacctcgcgaaaatccatggcgtgagcaaaaatgaactctacgatgcggttatttctgccagggaaaattaatcggatcccg